(19) United States
(12) Patent Application Publication
(10) Pub. No.: US 2002/0032193 A1
(43) Pub. Date: Mar. 14, 2002

(54) Use of potassium channel openers for the treatment of insulitis

(76) Inventors: John Bondo Hansen, Jyderup (DK); Anders Karlsson, Uppsala (SE); Mikael Kullin, Uppsala (SE); Stellan Sandler, Uppsala (SE); Elisabeth Bjork, Taby (SE); Zhanchun Li, Uppsala (SE); Birgitte Michelsen, Lyngby (DK); Jesper Svendstorp Lund, Copenhagen O (DK)

Correspondence Address: Reza Green, Esq., Novo Nordisk of North America, Inc., Suite 6400, 405 Lexington Avenue, New York, NY, 10174-6401, US

(21) Appl. No.: 09/891,692

(22) Filed: Jun. 26, 2001

Related U.S. Application Data
(60) Provisional application No. 60/217,902, filed on Jul. 13, 2000.

(30) Foreign Application Priority Data
Jun. 26, 2000 (DK) PA 2000 00988

(51) Int. Cl. A61K 31/549

(52) U.S. Cl. 514/222.8

(57) ABSTRACT

The present invention relates to the use of potassium channel agonists for the treatment of insulitis associated with various forms of diabetes such as IDDM, NIDDM, SPIDDM (LADA) and gestational diabetes.

### CROSS-REFERENCE TO RELATED APPLICATIONS

This application claims priority under 35 U.S.C. 119 of Danish application no. PA 2000 00988 filed on Jun. 26, 2000, and U.S. provisional application No. 60/217,902 filed on Jul. 13, 2000, the contents of which are fully incorporated herein by reference.

### FIELD OF THE INVENTION

The present invention relates to the use of potassium channel openers, which are able to protect the beta cells against toxic damage, for treating or preventing diseases related to autoimmune destruction of human beta cells, such as different types of diabetes, and methods of using these compounds.

### BACKGROUND OF THE INVENTION

Streptozotocin and alloxan are beta cell toxins. The toxic effect of these compounds on rat pancreatic islets in vitro and in vivo mimics the beta-cell death associated with Type 1 and late state Type 2 diabetes.

It has now been found that the compounds of the present invention are able to inhibit streptozotocin and alloxan induced beta cell degeneration and death.

The compounds of the present invention, known as potassium channel openers, act as activators of ATP regulated potassium channels (Katp-channels) of the beta cell and the Katp-channels of mitochondria. They may also act by antagonising the depletion of AND induced in the islets by these toxins. Cytokines are known to reduce beta cell viability and to induce apoptosis. Cytokines have been proposed to be involved with the autoimmune degeneration of beta cells in Type 1 diabetes. The compounds of the present invention antagonize the effects of cytokines on beta cells.

Thus, the compounds of the present invention can be used in the treatment of insulitis associated with various forms of diabetes.

Various forms of diabetes are Type 1 or Insulin Dependent Diabetes Mellitus (IDDM), Type 2 diabetes or Non Insulin Dependent Diabetes Mellitus (NIDDM), slowly progressive IDDM (SPIDDM) also referred to as latent autoimmune diabetes in adults (LADA) and gestational diabetes due to underlying IDDM.

Examples of potassium channel openers are compounds disclosed in PCT Publication No. WO 97/26265 (see for instance from page 14, line 5 to page 19, line 9) and WO 99103861 (see for instance from page 17, line 20 to page 19, line 5) as well as the following compounds: 3-tert-Butylamino-6-chloro-4H-thieno[3,2-e]-1,2,4-thiadiazine 1,1-dioxide; 6-Chloro-3-cyclobutylamino-4H-thieno[3,2-e]-1,2,4-thiadiazine 1,1-dioxide; 6-Chloro-3-(1,1-dimethylpropylamino)-4H-thieno[3,2-e]-1,2,4-thiadiazine 1,1-dioxide; 6-Chloro-3-(1-methylcyclopropyl)amino-4H-thieno[3,2-e]-1,2,4-thiadiazine 1,1-dioxide; 6-Chloro-3-(2-hydroxy-1,1-dimethylethylamino)-4H-thieno[3,2-e]-1,2,4-thiadiazine 1,1-dioxide and 6-Chloro-3-(1,1,3,3-tetramethylbutylamino)-4H-thieno[3,2-e]-1,2,4-thiadiazine 1,1-dioxide.

### DESCRIPTION OF THE INVENTION

The influence of ATP sensitive potassium (K_{ATP}) channel openers, diazoxide and a analogue, 6-Chloro-3-isopropylamino-4H-thieno[3,2-e]-1,2,4-thiadiazine 1,1-dioxide, has been examined on experimental beta-cell damage induced by streptozotocin (STZ), alloxan or cytokines. Rat islets were preincubated for 30 minutes with the K_{ATP} channel openers and subsequently incubated for 30 minutes following the addition of STZ. The islets were then washed and cultured for 24 hours. The STZ treatment (0.5 mM) was associated with a 40% islet loss. The remaining islets showed reduced insulin content and secretion and a reduced insulin biosynthesis, amounting to 50%, 60% and 35%, respectively of control. The STZ islets also displayed a lowered rate of glucose oxidation—16% of control. In contrast, islets pre-incubated with diazoxide or 6-Chloro-3-isopropylamino-4H-thieno[3,2-e]-1 ,2,4-thiadiazine 1,1-dioxide maintained higher insulin content and insulin secretion compared to islets incubated with STZ alone. In particular following incubation with 0.3 mM 6-Chloro-3-isopropylamino-4H-thieno[3,2-e]-1,2,4-thiadiazine 1,1-dioxide+STZ, there was no islet loss. In addition to having higher insulin content and secretion, these islets also had higher insulin biosynthesis and glucose oxidation rate than islets incubated with STZ alone. We also examined the influence of these K_{ATP} channel openers on damage induced by alloxan, a generator of reactive oxygen species. In these experiments, insulin release was reduced by 31% after treatment with 0.5 mM alloxan. This reduction was fully counteracted by simultaneous incubations with 0.3 mM 6-Chloro-3-isopropylamino-4H-thieno[3,2-e]-1,2,4-thiadiazine 1,1-dioxide or 0.3 mM diazoxide. Glucose oxidation rate in islets treated with 0.5 mM alloxan was decreased after 24 hours by 51%. Islets treated with alloxan in the presence of diazoxide had a glucose oxidation rate of 73% of control. Islets incubated with 6-Chloro-3-isopropylamino-4H-thieno[3,2-e]-1,2,4-thiadiazine 1,1-dioxide did not differ from control. The results demonstrate that K_{ATP} channel openers can protect insulin-producing cells from being damaged by a beta-cell toxin and suggest that such an effect might be applicable in subjects with ongoing insulitis.

Diazoxide and other K_{ATP} channel openers, such as cromakalim and pinacidil, have been employed in experimental studies of ischemic heart. A beneficial, cardioprotective effect was observed (Garlid KD et al., Circulation Res 1997; 81:1072-82). Although the mechanism of this phenomenon is not understood, an opening of mitochondrial potassium channels seems to be involved, resulting in dissipation of the inner mitochondrial membrane potential. This in turn leads to net oxidation of the mitochondria with an apparent reduction of energy wastage.

Diazoxide is known to act on K_{ATP} channels in the plasma membrane of beta cells. It hyperpolarizes the membrane and reduces the entry of Ca²⁺, essential for the exocytosis of secretory granulaes. Recently, exposure of beta cells to diazoxide was found to engage also mitochondrial K_{ATP} channels (Grimmsmann T et al., Br J Pharmacol 1998; 123:781-788). In the present study, we examined the influence of potassium channel openers on experimental beta-cell damage induced by streptozotocin, an agent known to cause energy depletion, on damage induced by alloxan, a generator of reactive oxygen species and on damage induced by cytokines.

Islet Isolation, Culture and Experimental Design

Pancreata from Sprague-Dawley rats were collagenase digested and islets collected with a braking pipette as previously described (Sandler S et al., Endocrinology, 1987;121:1424-31). Islets were precultured free floating in RPMI 1640 medium with 10% (v/v) fetal calf serum (FCS) and 11 mM glucose for 3 days in 5% CO₂ at 37° C. before experiments. Medium was changed two times during preculture. Islets were then transferred to sterile Petri dishes in KRBH (Krebs-Ringer bicarbonate with HEPES) medium with 2 mg/mL bovine serum albumin (BSA) and 5.6 mM glucose.

Stock solutions of test compounds dissolved in dimethyl sulphoxide were prepared and added to the Petri dishes. Islets were incubated in 5% CO₂ at 37° C. for 30 minutes with or without test compounds and STZ in 0.9% NaCl was then added to a final concentration of 0.5 mM. Dry powder of alloxan was diluted to a stock of 50 mM just before the addition to the Petri dishes to a final concentration of 0.5 mM. The incubation continued for another 30 minutes and was terminated by the addition of 1 mL of cold KRBH. The islets were then washed twice in KRBH and studied for morphology and insulin secretion, or cultured for 2 or 24 hours in RPMI with 10% FCS and 11 mM glucose prior to morphological and biochemical examinations.

Morphology and Islet Recovery

About 100 islets per condition were carefully transferred to a glass tube and spun down at 800 rpm for 1 minute. The medium was removed and about 200 μl left before the fixation with 8 ml of Bouin's medium, followed by dehydration in ethanol. The pellets were embedded in paraffin, cut in 5 μm sections and stained for insulin (guinea-pig anti-insulin, 1:100 dilution, DAKO, Sweden) using the PAP method. For estimation of islet recovery, 30 islets from each condition were transferred to Petri dishes as described above and the remaining islets counted after 24 hours.

Insulin Secretion and Islet Insulin Contents

Triplicates of five islets were transferred to 200 μl of KRBH with 2 mg/mL BSA and 16.7 mM glucose and incubated for 60 minutes in 5% CO₂ at 37° C. Islets from each condition were then pooled and sonicated in 200 μl of redestilled water. A 50 μl aliquot of the homogenate was mixed with 125 μl acid ethanol (0.18 M HCl in 95% ethanol) and insulin extracted overnight. Insulin concentration in the sonicate and the culture medium was determined with radioimmunoassay.

Proinsulin Biosynthesis and Total Protein Biosynthesis

For each condition duplicate samples of 20 islets were transferred to multiwell plates containing 100 μl KRBH with L-[4.5-³H]leucine (50 μCi/ml), 2 mg/mL BSA and 16.7 mM glucose and incubated for 120 minutes in 5% C0₂ at 37° C. Islets were then washed in Hanks' solution supplemented with 10 mM nonradioactive leucine and subsequently sonicated in 200 μl of redestilled water. A 50 μl fraction of the aqueous homogenate was incubated for 90 minutes with insulin antibodies coupled to Sepharose beads to separate proinsulin from other labelled proteins (15). Total protein biosynthesis was obtained by precipitating the labelled proteins with trichloroacetic acid (TCA). The antibody bound and TCA precipitable radioactivity were determined in a liquid scintillation counter.

Glucose oxidation

Groups of 10 islets were transferred to glass vials with 100 μl KRBH supplemented with D-[U¹⁴C]glucose and nonradioactive glucose to a final concentration of 16.7 mM glucose. Triplicate samples were used. The vials were suspended in scintillation flasks, gassed with 5% CO₂ and sealed airtight. The flasks were then shaken for 90 minutes at 37° C. Metabolism was stopped by injection of 100 μl of 0.05 mM antimycin A into the center vial. Immediately thereafter 250 μl hyamine hydroxide was injected into the outer flask. CO₂ was released from the incubation medium by injecting 100 μl of 0.4 M Na₂HPO₄ solution (pH 6.0) into the center vial. To allow the CO₂ to be trapped by the hyamine hydroxide the vials were incubated for another 120 minutes at 37° C. Scintillation fluid was then added to each flask and the radioactivity counted in a liquid scintillation counter.

Statistics

Students' paired t-test and analysis of variance (ANOVA) were used when appropriate.

Islet Recovery and Morphology

The islets exposed to Streptozotocin for 30 minutes showed degranulation, and in some islets numerous pyknotic nuclei, at the 0 hour timepoint. No signs of recovery but a further destruction and also disintegration of islets was found at 2 and 24 hours. In contrast, islets incubated with test compounds+STZ appeared morphologically intact at the 0 hour timepoint. During the subsequent 24 hour culture a toxic effect of STZ became noticeable. At 2 hours the surface of these islets were somewhat irregular and this was more apparent at 24 hours. The numerous pyknotic nuclei as seen in the STZ group were not found in the group of islets treated with test compounds.

Islets examined at the 0 hour timepoint, ie after a 60 minutes incubation in 5.6 mM glucose, showed a stronger stain for insulin than the islets examined after 2 and 24 hours. The latter islets had been cultured in 11 mM glucose. The difference in insulin staining reflects a higher stimulation of insulin secretion at 11 mM compared to 5.6 mM glucose. The insulin staining of the islets treated with test compounds+streptozotocin were stronger at both 2 and 24 hours than that seen with the islets incubated with medium alone.

Functional Characteristics

The islets recovered 24 hours after the STZ treatment had reduced insulin content and glucose-stimulated insulin release. The STZ treatment also had lowered the insulin and total protein biosynthesis as well as impaired the glucose oxidation rate. An inhibition of insulin secretion was found with islets incubated with test compounds alone at 0 and 2 hours but not at 24 hours. The inhibitory effect of the KATP channel openers on insulin secretion was seen in islets treated with test compounds+streptozotocin at 0 and 2 hours, but not after 24 hours. At 24 hours following test compounds+STZ treatments, a partial protection of the islet function was observed when compared with islets incubated with STZ alone.

At 24 hours, the proinsulin and total protein biosynthesis in the recovered STZ islets were reduced to 35% and 51% of control, respectively. The lowering of the proinsulin/total protein biosynthesis ratio, 15% compared to 23% in control islets, indicates a preferential beta-cell effect of the STZ treatment. In islets treated with test compounds+streptozotocin the proinsulin and total protein biosynthesis did not differ from the biosynthesis found in the recovered STZ.

Cytokine induced beta cell toxicity

The effect of PCO compounds on cell viability was analysed in ⁵¹Cr-release cytotoxicity assays using either primary islet preparations (e.g. from newborn rats) or islet tumour cell lines (e.g. mouse transgenic β-cell lines βTC-3 or Min6, or rat insulinoma lines RIN5AH or MSLG2). The assay has been used to measure toxic effects of e.g. cytokines or glucose, and to address the protective effect of PCO compounds on β-cell viability, e.g. during cytokine exposure.

### METHODS

Viability assay using primary islets:

Approximately 3500 islets were washed and resuspended in 15 ml islet media (RPMI1640 (Life tech cat 61870-010)+10% FCS (Life cat 16000-044))+100 lU/ml Penicillin 100 UG/ ml streptomycin). 2,5 μCi/mi Na⁵¹Cr (Dupont, Nez 030S) was added and the suspension was transferred to a 60 mm petri dish and incubated overnight at 37° C. and 5% C0₂. After incubation the islets were washed 3 times in 1×HBSS (life tech without Ca⁺⁺ and Mg⁺⁺ Cat 14185-045). The islets were then resuspended in 10 ml Islet media and 100 μl of the islet suspension were added to each well in a flat bottom 96 well plate (approximately 35 islets in each well). Mixture of cytokines and test compounds or dimethyl sulphoxide were prepared in 100 μl media in each well. All test compounds were dissolved in dimethyl sulphoxide and prepared in stock solutions at a concentration of 100mM. Stock solutions of 10 ng/μl of cytokines (Pharmingen mrlL-1 β; 19201V, mrTNF-α; 19321T; mrlFN-γ, 19301T) dissolved in distilled H₂O were prepared, and added to the wells in final concentrations ranging from 0.01 ng/ml to 100 ng/mi.

The islets were incubated for 48h at 37° C. and 5% CO₂. The plates were centrifuged for 5 min at 1000 rpm, and 100 μl supernatant samples were harvested from each well. 100 μl 1% triton-X were added to each well in order to lyse the islets and 100 μl were harvested to obtain the total releasable Na⁵¹Cr from the islets of each well. All the samples and the maximum samples were counted on a Cobra γ-counter (Packard). The release of Na⁵¹Cr was calculated for each sample, by normalizing to its own maximum and calculated by the following equation: ((Sample in %-spontaneous in %)/(100-spontaneous))%. All samples were made in quadruplicates.

Normalised sample=(Sample cpm/(sample maximum*2))*100%

Spontaneous release=(Untreated cells cpm/(sample maximum*2))*100%

Viability assay using rodent adherent β-cell lines (e.g. RIN cells, MIN6 cells, Ins-1 cells and others)

Cells were grown to approximately 80% confluence. After washing once in HBSS (life tech without Ca⁺⁺ and Mg⁺⁺ Cat 14185-045), 1×trypsin in HBSS was used to split the cells. The cells were seeded in a flat-bottomed 96 well plate in the desired media at a density of 40000 cells/well in 100 μl media and incubated overnight to secure proper adherence. 2.5 μCi/ml Na⁵¹Cr (Dupont, Nez 030S) was added to the labeling media (the desired media). After 1× washing of the cells with HBSS 200 μl of media with Na⁵¹Cr were added to each well and incubated overnight. After Na⁵¹Cr incubation cells were washed twice in HBSS, before addition of media with cytokines and PCO compounds or dimethyl sulphoxide. Mixture of these media was prepared in stocks with 200 μl for each well. All PCO-compounds were dissolved in dimethyl sulphoxid and prepared in stock solutions at a concentration of 100 mM. Stock solutions of 10 ng/μl, of cytokines (Pharmingen mrlL-1β; 19201V, mrTNF-α; 19321T; mrlFN-γ, 19301T) dissolved in distilled H₂O were prepared, and added to the stocks in final concentrations ranging from 0.1 ng/ml to 100 ng/ml.

The rodent adherent β-cell lines were incubated for 24 h at 37° C. and 5% CO₂. The plates were centrifuged for 5 min at 1000 rpm, and 100 μl supernatant samples were harvested from each well. 100 μl 1% triton-X were added to each well in order to lyse the cells and 100 μl were harvested to get a maximum Na⁵¹Cr release from the cells of each well. All the samples and the maximum samples were counted on a cobra γ-counter (Packard). The release of Na⁵¹Cr was calculated for each sample, by normalizing to its own maximum and calculated by the following equation: ((Sample in %-spontaneous in %)/(100-spontaneous))%. All samples were made in quadruplicates.

Normalised sample=(Sample cpm/(sample maximum 2)) 100%

Spontaneous release=(Untreated cells cpm/(sample maximum 2)) 100%

Effects on mitochondria.

The effects on mitochondrial Katp channels kan be evaluated as described by e.g. Grimmsmann and Rustenbeck (Br. J. Pharmacol. 1998, 123, 781-788). Routinely the effects of the compounds of the present invention can be determined measuring changes in fluorescence of the dyes JC-1 or Rhodamine 123 when incubating beta cells or pancreatic islets in a medium containing the fluorencence indicators and the test compounds.

What is claimed is:

1. The use of a potassium channel opener protecting the beta cells against toxic damage for the preparation of a pharmaceutical composition for treating or preventing diseases related to autoimmune destruction of human beta cells.

2. The use according to claim 1 wherein the protection of the beta cells is established through an opening of mitochondrial potassium channels.

3. The use according to anyone of the preceding claims wherein the diseases are related to different types of diabetes selected from the group consisting of IDDM, NIDDM, SPIDDM or LADA and gestational IDDM.

4. The use according to anyone of the preceding claims wherein the potassium channel opener is selected from:
6-Chloro-3-isopropylamino-4H-thieno[3,2-e]-1,2,4-thiadiazine 1,1-dioxide,
3-tert-Butylamino-6-chloro-4H-thieno[3,2-e]-1,2,4-thiadiazine 1,1-dioxide,
6-Chloro-3-(1,1-dimethylpropylamino)-4H-thieno[3,2-e]-1,2,4-thiadiazine 1,1-dioxide,
6-Chloro-3-(1-methylcyclopropyl)amino-4H-thieno[3,2-e]-1,2,4-thiadiazine 1,1-dioxide,
6-Chloro-3-(2-hydroxy-1,1-dimethylethylamino)-4H-thieno[3,2-e]-1,2,4-thiadiazine 1,1-dioxide,
6-Chloro-3-(1,1,3,3-tetramethylbutylamino)-4H-thieno[3,2-e]-1,2,4-thiadiazine 1,1-dioxide, or other potassium channel openers as disclosed in the description.

5. The use of a potassium channel opener antagonising streptozotocin induced depletion of AND in the pancreatic islets for the preparation of a pharmaceutical composition for treating or preventing diseases related to autoimmune destruction of human beta cells.

6. The use according to claim 5 wherein the depletion of AND in the pancreatic islets is obtained through inhibition of poly(ADP-ribose)synthetase.

7. The use according to claim 5 or 6 wherein the diseases are related to different types of diabetes selected from the group consisting of IDDM, NIDDM, SPIDDM or LADA and gestational IDDM.

8. The use according to anyone of the preceding claims 5-7 wherein the potassium channel openers is selected from:
6-Chloro-3-isopropylamino-4H-thieno[3,2-e]-1,2,4-thiadiazine 1,1-dioxide,
3-tert-Butylamino-6-chloro-4H-thieno[3,2-e]-1,2,4-thiadiazine 1,1-dioxide,
6-Chloro-3-(1,1-dimethylpropylamino)-4H-thieno[3,2-e]-1,2,4-thiadiazine 1,1-dioxide,
6-Chloro-3-(1-methylcyclopropyl)amino-4H-thieno[3,2-e]-1,2,4-thiadiazine 1,1-dioxide,
6-Chloro-3-(2-hydroxy-1,1-dimethylethylamino)-4H-thieno[3,2-e]-1,2,4-thiadiazine 1,1-dioxide,
6-Chloro-3-(1,1,3,3-tetramethylbutylamino)-4H-thieno[3,2-e]-1,2,4-thiadiazine 1,1-dioxide, or other potassium channel openers as disclosed in the description.

9. A method of treating or preventing diseases related to autoimmune destruction of human beta cells comprising administering a potassium channel operner in an amount effective to protect the beta cells against toxic damage.

10. A method according to claim 9 wherein the protection of the beta cells is established through an opening of mitochondrial potassium channels.

11. A method according to claim 9 wherein the diseases are related to different types of diabetes selected from the group consisting of: IDDM, NIDDM, SPIDDM or LADA, and gestational IDDM.

12. A method according to claim 9 wherein the potassium channel opener is selected from the group consisting of:
6-Chloro-3-isopropylamino-4H-thieno[3,2-e]-1,2,4-thiadiazine 1,1-dioxide,
3-tert-Butylamino-6-chloro-4H-thieno[3,2-e]-1,2,4-thiadiazine 1,1-dioxide,
6-Chloro-3-(1,1-dimethylpropylamino)-4H-thieno[3,2-e]-1,2,4-thiadiazine 1,1-dioxide,
6-Chloro-3-(1-methylcyclopropyl)amino-4H-thieno[3,2-e]-1,2,4-thiadiazine 1,1-dioxide,
6-Chloro-3-(2-hydroxy-1,1-dimethylethylamino)-4H-thieno[3,2-e]-1,2,4-thiadiazine 1,1-dioxide, and
6-Chloro-3-(1,1,3,3-tetramethylbutylamino)-4H-thieno[3,2-e]-1,2,4-thiadiazine 1,1-dioxide.

13. A method of treating or preventing diseases related to autoimmune destruction of human beta cells in pancreatic islets, comprising administering a potassium channel opener in an amount effective to antagonize streptozotocin-induced depletion of AND in the pancreatic islets.

14. A method according to claim 13 wherein the depletion of AND in the pancreatic islets is obtained through inhibition of poly(ADP-ribose)synthetase.

15. A method according to claim 13 wherein the diseases are related to different types of diabetes selected from the group consisting of: IDDM, NIDDM, SPIDDM or LADA, and gestational IDDM.

16. A method according to claim 13 wherein the potassium channel opener is selected from the group consisting of:
6-Chloro-3-isopropylamino-4H-thieno[3,2-e]-1,2,4-thiadiazine 1,1-dioxide,
3-tert-Butylamino-6-chloro-4H-thieno[3,2-e]-1,2,4-thiadiazine 1,1-dioxide,
6-Chloro-3-(1,1-dimethylpropylamino)-4H-thieno[3,2-e]-1,2,4-thiadiazine 1,1-dioxide,
6-Chloro-3-(1-methylcyclopropyl)amino-4H-thieno[3,2-e]-1,2,4-thiadiazine 1,1-dioxide,
6-Chloro-3-(2-hydroxy-1,1-dimethylethylamino)-4H-thieno[3,2-e]-1,2,4-thiadiazine 1,1-dioxide, and
6-Chloro-3-(1,1,3,3-tetramethylbutylamino)-4H-thieno[3,2-e]-1,2,4-thiadiazine 1,1-dioxide.
